(19) United States
(12) Patent Application Publication
(10) Pub. No.: US 2009/0304618 A1
(43) Pub. Date: Dec. 10, 2009

(54) ACYL LACTYLATE COMPOSITIONS FOR RINSE-OUT AND LEAVE-ON APPLICATIONS FOR SKIN AND HAIR

(75) Inventors: Irene Shapiro, Buffalo Grove, IL (US); Gregory Dado, Chicago, IL (US); David Allen, Oak Brook, IL (US); Branko Sajic, Lincolnwood, IL (US)

Correspondence Address: MCANDREWS HELD & MALLOY, LTD, 500 WEST MADISON STREET, SUITE 3400, CHICAGO, IL, 60661, US

(73) Assignee: Stepan Company, Northfield, IL (US)

(21) Appl. No.: 12/162,886

(22) PCT Filed: Feb. 27, 2007
(86) PCT No.: PCT/US07/05001

§ 371 Date: Oct. 28, 2008

Related U.S. Application Data
(60) Provisional application No. 60/777,001, filed on Feb. 27, 2006.

(51) Int. Cl. A61K 8/37; A61Q 5/00; A61Q 19/00; C11D 9/00; C11D 17/00; C11D 3/20

(52) U.S. Cl. 424/70.1; 514/547; 510/159; 510/119; 510/129; 510/137; 510/135; 510/152

(57) ABSTRACT

The presently described technology relates to acyl lactylate containing compositions having improved stability and multifunctional performance characteristics (e.g., pH stability, phase stability, hydrolytic stability (e.g., aqueous stability), cleansing, foaming, skin softness and skin moisturization) that may be utilized in rinse-out and leave-on skin and hair applications. The acyl lactylate compositions of the present technology may be delivered to the skin or hair via a conventional delivery vehicle such as a body wash, a shampoo, a bubble bath, a skin cream, or a lotion.

### RELATED APPLICATIONS

This application makes reference to, claims priority to and claims the benefit of U.S. provisional patent application No. 60/777,001, filed on Feb. 27, 2006.

### FIELD OF THE INVENTION

In general, the presently described technology relates to acyl lactylates and compositions containing such acyl lactylates having improved stability characteristics (e.g., pH stability, phase stability, and hydrolytic stability) that may be utilized in rinse-out and leave-on skin and hair applications such as a body wash, shampoo, liquid hand soap, skin cream, lotion, and the like. In particular, the acyl lactylate compositions provide improved multifunctional performance properties including foaming, cleansing, and skin feel (e.g., softness and moisturization) after application to skin or hair.

### BACKGROUND OF THE INVENTION

In the recent past, it has become increasingly important to consumers that cleansing, foaming compositions (either in a rinse-out or a leave-on delivery vehicle) exhibit high foaming capability while still maintaining mild outcomes to skin and hair. Consequently, efforts have been made to provide products, such as hair shampoos, shower gels, lotions, facial cream, and facial wash foams, showing these two properties. However, at least one of the major problems associated with such products is the difficulty to combine the desired properties into one singular product. Typically, high lathering surfactants are generally very harsh to the skin and hair, while mild surfactants tend to give insufficient lather. As a result, conventionally available rinse-out or leave-on skin and hair treatments tend to inadequately provide the combined cleansing and foaming effect desired. Further, additional desired multifunctional properties such as skin softness and skin moisturization are also hindered by the formulation incompatibility of the components used in an effort to impart the desired properties to the resultant final composition.

In the past, attempts have been made to overcome these problems by combining harsh surfactants (that generate sufficient lather) with very mild co-surfactants. Such outcomes have not achieved customer satisfaction, product acceptance, or the ability to impart the desired multifunctional properties to skin and hair through one delivery vehicle.

For example, U.S. Pat. No. 3,728,447 (C J Patterson) discloses hair shampoo compositions containing fatty acid lactylates or glycolates. While the cleaning action of these shampoos is described as being satisfactory, the resultant foam remained minimal. In order to achieve higher foaming action in the described shampoo compositions, the reference indicates that additional (and often expensive) booster surfactants, such as triethanol amine lauryl sulphate, should be incorporated. However, such booster surfactants often cause a high degree of skin irritation.

WO 91/09923 (Farris, et al.) describes compositions that comprise an ultra mild surfactant and a foam enhancer. The ultra mild surfactant is an alkyl glyceryl ether sulfonate (AGS) having a hydrophobic group containing a linear alkyl chain containing from about 7 to 9 carbon atoms. Conventional foam boosters, such as amine oxides and water soluble halide salts, are added to improve lather creaminess, volume and stability. Again, such foam boosters can be harsh to the skin and are expensive to incorporate into the final resultant products.

EP 0 224 796 (Kao) describes a detergent composition comprising: (a) a phosphate surfactant and (b) an acyl lactylate having an acyl group containing 12 to 18 carbon atoms. Specific examples for such combinations are mixtures of lauryl phosphate and stearoyl lactylate, isostearoyl lactylate and lauroyl lactylate, respectively.

U.S. Pat. No. 4,761,279 (Eastman Kodak) discloses shaving cream formulations comprising salts of C₁₄-C₂₂ acyl lactylates, saturated monoglycerides, propylene glycol monoesters and humectants.

U.S. Pat. No. 6,001,787 (Pratley) describes a combination of specific short-chain surfactants with specific long-chain surfactants to provide mild cleansing, high foaming skin and hair care compositions. Such short-chain and long-chain surfactant compositions are expensive to manufacture.

Acyl lactylates have been used in personal care formulations to provide skin-feel and hair care benefits. However, it is also known that such components can exhibit hydrolysis in aqueous-based formulations, resulting in a poor shelf-life of the composition. In addition, hydrolysis can cause an increase in acidity of the composition which can be harsh on the skin. Additional components can be added to the formulations to reduce the effect of hydrolysis, but such additional components increase the cost of the formulation.

There is, therefore, a need in the cosmetic and personal care fields for a cost-effective rinse-out or leave-on skin or hair aqueous-based composition(s) containing an acyl lactylate of the present technology described herein that achieves superior cleaning and foaming properties in a stable manner (e.g., phase stability, hydrolytic stability (preferably in an aqeuous environment), pH stability, etc.) while imparting improved multifunctional performance properties (e.g., softness and moisturization) to skin and/or hair unlike currently available personal care and cosmetic compositions, in particular those that are aqeuous-based. Further, the ability of such components of the present technology to deliver multifunctional properties permits the use of fewer components in end-use cosmetic or personal care products which, in turn, results in lower costs of production. Additionally, the use of such components can also lead to improved shelf-life for those end-use cosmetic and personal care compositions as well.

It has now been surprisingly discovered that cleansing and high foaming can be achieved in rinse-out and leave-on skin and hair applications such as a body wash, bubble bath, shampoo, skin cream, or lotion through the use of the particular acyl lactylate compositions of the presently described technology. Unexpectedly, cleansing and foaming properties have been achieved by combining the acyl lactylate with other surfactants, which together achieve stable compositions that impart improved multifunctional performance properties to the skin and hair.

### BRIEF SUMMARY OF THE INVENTION

The presently described technology relates to acyl lactylate compositions having improved stability characteristics (e.g., hydrolytic stability (such as aqueous hydrolytic stability), pH stability and phase stability) and improved multifunctional performance properties (e.g., skin and/or hair softness and moisturization) that may be utilized in rinse-out and leave-on skin and hair applications such as a body wash, shampoo, liquid hand soap, bubble bath, skin cream, lotion, and the like. The improved properties are achieved by utilizing acyl lactylate compositions that minimize the amount of lactylic acid present in the acyl lactylate compositions compared to known acyl lactylate compositions. Preferably the amount of lactylic acid present in the acyl lactylate compositions is no greater than about 2.0% by weight of the acyl lactylate composition.

In one embodiment there is provided a rinse-out skin or hair composition comprising at least one acyl lactylate composition, wherein the acyl lactylate composition has an amount of lactylic acid present of no more than about 2.0% by weight of the acyl lactylate composition, at least one surfactant, and water.

In another embodiment there is provided a soap bar composition comprising at least one acyl lactylate composition, wherein the acyl lactylate composition has an amount of lactylic acid present of no more than about 2.0% by weight of the acyl lactylate composition, at least one surfactant, and water.

Further embodiments of the present technology may additionally incorporate fragrances, dyes, proteins, preservatives, opacifying agents, pearlescent agents, thickeners, herbal extracts, vitamins and the like.

While the presently described technology will be described in connection with one or more preferred embodiments, it will be understood by those skilled in the art that it is not limited to those embodiments. On the contrary, the presently described technology includes all alternatives, modifications and equivalents as may be included within the spirit and scope of the appended claims.

### BRIEF DESCRIPTION OF THE DRAWINGS

FIG. 1 is a graph illustrating the pH stability of the acyl lactylate composition of the present technology in a sodium surfactant system.

FIG. 2 is a bubble chart comparing the skin-feel properties of a composition in accordance with the present technology with the skin-feel properties of a control composition and competitive compositions.

FIG. 3 is a bubble chart comparing the skin-feel properties of a composition in accordance with the present technology with the skin-feel properties of compositions prepared with polymeric additives on an equal cost basis.

FIG. 4 is a bar graph comparing the tackiness properties of a composition in accordance with the present technology with the tackiness properties of compositions prepared with polymeric additives on an equal cost basis.

FIG. 5 is a bar graph comparing the performance properties of a composition in accordance with the present technology with the performance properties of a control composition.

### DETAILED DESCRIPTION OF THE INVENTION

The skin and hair compositions of the presently described technology comprise at least one acyl lactylate composition having improved stability (e.g., hydrolytic stability (preferably aqueous hydrolytic stability)), pH stability, and/or phase stability as well as multifunctional performance characteristics (e.g., cleansing, foaming, skin softness and skin moisturization), at least one surfactant, and water.

Acyl lactylate compositions are commercially available and have been used in food and personal care compositions. Such acyl lactylate compositions are typically prepared by reacting a fatty acid and lactic acid with sodium hydroxide (NaOH) in accordance with the following reaction scheme:

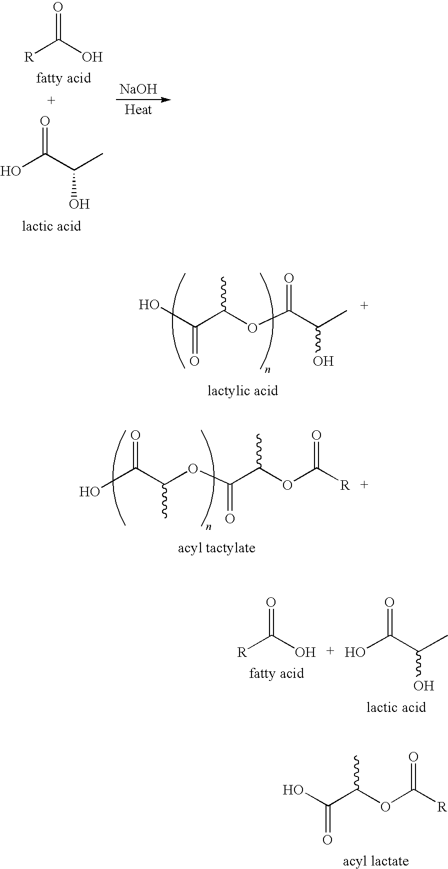

The resulting acyl lactylate composition actually comprises a mixture of the components lactylic acid (n≧1), fatty acid, lactic acid (n=0), acyl lactylate (n>1) and acyl lactate (n=1). Preferably, the acid portions of the acyl lactylate compositions of the present technology are at least partially neutralized (e.g., as sodium salts). Further, the components comprising the acyl lactylate composition are present in the composition in varying amounts, depending upon the reaction stoicheometry. In general, however, commercially available acyl lactylate compositions comprise from about 37% to about 53% by weight of the acyl lactate component (n=1), from about 20% to about 25% by weight of the fatty acid component, from about 11% to about 32% by weight of the acyl lactylate component (n>1), from about 4% to about 13% of the lactic acid component, and greater than 2% of the lactylic acid component. Sources for such commercially available acyl lactylates include American Ingredients Company, Kansas City, Mo., RITA Corporation, Crystal Lake, Ill., Kerry Bio Science, Hoffman Estates, Ill., and Abitec, Ltd., Northampton, England.

Aqueous-based compositions that contain at least one of the above-described commercially available acyl lactylate compositions are known to be hydrolytically unstable. While not wishing to be bound by any particular theory, it is believed that the lactylic acid component in the acyl lactylate composition causes hydrolysis of the ester components. Ester hydrolysis leads to a drop in pH which in turn increases the rate of ester hydrolysis, resulting in a hydrolytically unstable product.

It has been unexpectedly determined that by minimizing the amount of the lactylic acid component in the acyl lactylate composition, the resulting formulated end use products comprising the acyl lactylate compositions described herein have improved hydrolytic stability, improved pH stability, and improved viscosity stability at 50° C. as a function of time as compared to current and commercially available acyl lactylate compositions, namely those commercial applications containing acyl lactylate compositions having an amount of lactylic acid greater than 2.0% by weight of the acyl lactylate. It should be understood that the terms “acyl lactylate” and “acyl lactylate composition” as used herein refer to a mixture of the components lactylic acid (n≧1), fatty acid, lactic acid (n=0), acyl lactylate (n>1) and acyl lactate (n=1) resulting from the reaction of a fatty acid and lactic acid, unless the context indicates otherwise.

The lactylic acid component is preferably present in the acyl lactylate composition in an amount no greater than about 2.0% by weight of the acyl lactylate composition and more preferably no greater than about 1.0% by weight. It is also desirable to minimize the amount of acyl lactylate (n>1) component present in the acyl lactylate composition(s) of the present technology and to maximize the amount of acyl lactate (n=1) component present in the same composition(s). A preferred amount of the acyl lactylate component is about 15% by weight or less, while a preferred amount of the acyl lactate component is at least about 50% or more.

Preferred and desirable amounts of the acyl lactylate components are obtained by controlling the stoicheometry of the starting reactants/components. The ratio of lactic acid to fatty acid in the reaction charge is preferably about 1.10:1, alternatively about 1:1, alternatively about 0.9:1, alternatively about 0.8:1. The degree of neutralization obtained via NaOH addition is desirably at least about 75% or greater, alternatively at least about 80% or greater.

The acyl group of the acyl lactylate composition can, preferably, be linear or branched and contains from 10 to 18 carbon atoms. Suitable examples of the acyl lactylate composition include lauroyl lactylate, stearoyl lactylate, isolauroyl lactylate, isostearoyl lactylate, myristoyl lactylate, isomyristoyl lactylate, and salts thereof. Suitable salts include, for example, alkali metal salts (e.g., sodium salts), ammonium salts, and amine salts. The most preferred acyl lactylate compositions for use herein are sodium lauroyl lactylate having a lactylic acid component of about 2.0% by weight or less, and sodium stearoyl lactylate having a lactylic acid component of about 2.0% by weight or less. Such acyl lactylate compositions are available from Stepan Company, Northfield, Ill. under the tradenames STEPAN® SLL-FB, and STEPAN® SSL-CG, respectively.

The acyl lactylate composition is combined with at least one other surfactant to formulate end use products including, but not limited to, a body wash, a shampoo, a liquid hand soap, a liquid facial cleanser, a liquid body scrub, a bubble bath, a skin cream of any nature (e.g., facial, hand or body) and a lotion. The surfactant comprises from about 0.5 to about 25 weight percent active of the formulated composition, and the acyl lactylate comprises from about 0.5 to about 25 weight percent active of the formulated composition. The total combination of acyl lactylate and surfactant comprises from about 3 to about 40 weight percent active of the formulated composition.

In the cleansing, foaming, rinse-out applications of the present technology, the acyl lactylates may be combined with other anionic, amphoteric, non-ionic and zwitterionic surfactants and water to formulate end use products (e.g., shampoo or body wash) which deliver multifunctional performance properties including, for example, foaming, cleansing, and skin feel after use (e.g., softness and moisturization).

Suitable anionic surfactants, include, without limitation: sulfonated alkyl benzene, sulfonated methyl esters, sulfonated alpha olefin, paraffin sulfonate, alkyl sulfate, alkyl alkoxy sulfate, alkyl alkoxy carboxylate, alkyl phosphate, alkyl alkoxy phosphate, alkyl sulfonate, alkyl alkoxylated sulfonate, alkyl isethionate, salts thereof, and combinations thereof. Further examples can be found in “Surface Active Agents and Detergents” (Vol. I and II by Schwartz, Perry and Berch). Suitable nonionic surfactants include, without limitation: fatty acid amide, ethoxylated fatty acid amide, alkyl alcohol, alkyl alcohol ethoxylate, alkyl phenol ethoxylate, propylene glycol esters, polyglycerol esters, ethylene glycol esters, ethoxylated glycol esters, polypropylene glycol esters, alkylpolyglycoside, alkyl glucamide, and combinations thereof. More examples are generally disclosed in U.S. Pat. No. 3,929,678 to Laughlin et al., issued on Dec. 30, 1975 at column 13, line 14 through column 16, line 6, incorporated herein by reference. Suitable amphoteric and zwitterionic surfactants include, without limitation, betaines, amine oxides, amphoacetates, amphodipropionates, and amphocarboxyglyconates.

Further, other additives such as fragrances, dyes, proteins, preservatives, opacifying agents, thickeners, pearlescent agents and others can also be added to the formulated products. Thus, one of ordinary skill in the art will appreciate that the acyl lactylate containing compositions of the present technology may be formulated in a variety of ways necessary to suit the particular end use application.

The acyl lactylate compositions of the presently described technology can also be incorporated into combination soap bars with high moisture content (from about 5% to about 20% water, more preferably from about 8% to about 20% water) to deliver skin softness and moisturization properties.

In emulsion treatment leave-on skin cream and lotion applications, the acyl lactylate compositions of the present technology can be combined with other co-emulsifiers (e.g., glycerol monostearate, glycerol stearate, cetyl alcohol, propoxylated fatty alcohol ethers, polyglycerol fatty esters, among others, except cationic types), emollient oils (e.g., white petrolatum, silicone oils, soybean oil, mineral oil, glycerin, among others), humectants, preservatives, dyes, thickeners or water, to formulate such creams and lotions. The resulting formulated products have better pH stability, phase stability at 50° C., higher viscosity and smaller particle size distribution that affords improved emulsion stability. Further, Example 4 as described herein provides further insight regarding an emulsion skin lotion utilizing the present technology.

The compositions and the methods of producing such compositions herein may be formulated and carried out such that they will have a pH of between about 4.0 to about 8.5, preferably, between about 5.0 to about 7.0. Techniques for controlling pH at recommended usage levels include the use of buffers, alkali, acids, etc., and are well known to those skilled in the art. Optional pH adjusting agents can include, but are not limited to citric acid, succinic acid, phosphoric acid, sodium hydroxide, sodium carbonate, and the like.

All documents, e.g., patents and journal articles, cited above and/or below, are hereby incorporated by reference in their entirety. In the following Examples, all amounts are stated as percent weight for each of the ingredients unless stated otherwise or provided for in parentheses. In the case of components being further described in parentheses of the examples herein, such amounts are based upon percent active material rather than weight percent. One skilled in the art will recognize that modifications may be made in the presently described technology without deviating from the spirit or scope of the invention. The presently described technology is also illustrated by the following examples, which are not to be construed as limiting the invention or scope of the specific procedures or compositions described herein. All levels and ranges, temperatures, results, etc., used and/or described herein are approximations unless otherwise specified.

### EXAMPLES

### Example 1

### Body Wash

| Phase | INCI (CTFA) Name/Chemical Name | Trade Name/Supplier Name | % | |
|---|---|---|---|---|
| 1 | Deionized Water | | Q.S. to 100.0 | |
| | Sodium C14-16 Olefin Sulfonate | BIO-TERGE ® AS-40 CG | 15.0 | (3-30% active) |
| | Ammonium Lauryl Sulfate | STEPANOL ® AM | 21.4 | (2-50% active) |
| | Cocamide MEA | NINOL ® COMF | 2.0 | (0.1-10% active) |
| | STEPAN ® SSL-CG (Sodium Stearoyl | | 2.0 | (0.1-20% active) |
| | Lactylate) | | | |
| | Glycol Stearate (And) Stearamide AMP | STEPAN ® EGAS | 1.0 | (0.1-10% active) |

| | |
|---|---|
| Borage Oil (Roche) | 0.1 |
| Preservative, Dye, Color | q.s. |
| Sodium Hydroxide 50% solution | q.s. |
| Citric Acid 50% solution | q.s. |
| Sodium Chloride | q.s. |

The values for each material are provided in terms of weight percent. However, the values noted in parentheses indicate the range for each material in terms of percent active ingredient. Thus, it should be understood by those skilled in the art that the Examples provided herein may be expanded based upon the percentage range of each component material or percent active ingredient noted within the respective formulation.

### Procedure for the Manufacture of Example 1:

1. Charge vessel with Deionized Water. Start agitation.
2. Add BIO-TERGE AS-40, STEPANOL AM and NINOL COMF. Start mixing and heating to 160-165° F.
3. At 160-165° F. add STEPAN SSL-CG and STEPAN EGMS, mix until dispersed. Keep mixing at this temperature for 20-25 minutes.
4. Start cooling to 80° F.

### 5. At 80° F., add Borage Oil mix.

6. Add preservative, dye, color, if desired. Mix well.
7. Adjust pH to 5.5-6.5 with Sodium Hydroxide or Citric Acid, as necessary.
8. Adjust viscosity with Sodium Chloride if necessary.

The resulting composition delivers a creamy, lubricating foam and leaves a velvety skin after-feel.

### Example 2

### Moisturizing Body Wash

| Phase | INCI (CTFA) Name/Chemical Name | Trade Name/Supplier Name | % | |
|---|---|---|---|---|
| 1 | Deionized Water | | Q.S. to 100.0 | |
| | Sodium Laureth Sulfate | STEOL ® CS-230 | 23.0 | (2-30% active) |
| | Sodium Lauryl Sulfate | STEPANOL ® WA-EXTRA | 20.0 | (2-30% active) |
| | Cocamidopropyl Betaine | AMPHOSOL ® HCG | 9.8 | (0.5-20% active) |
| | Glycerin (Dow) | | 0.5 | (0.1-10% active) |
| | STEPAN ® SSL-CG (Sodium Stearoyl Lactylate) | | 2.0 | (1-20% active) |

| | |
|---|---|
| Flax Extract (Natunola) | 1.0 |
| Sodium Hydroxide 50% solution | q.s. |
| Citric Acid 50% solution | q.s. |
| Sodium Chloride | q.s. |
| Preservative, Dye, Fragrance | q.s |

### Procedure:

1. Charge vessel with Deionized Water. Start agitation.
2. Add STEOL CS-230, STEPANOL WA-Extra, AMPHOSOL HCG and Glycerin. Mix until homogeneous. Start heating to 160-165° F.
3. At temperature 160-165° F. add STEPAN® SSL. Mix until completely dispersed. Keep mixing for 20 minutes.
4. Start cooling to 80° F.
5. At room temperature add Flax Extract. Mix.
6. Add preservative, fragrance and dye, if desired.
7. Adjust pH to 5.5-6.5 with Sodium Hydroxide or Citric Acid, if necessary.
8. Adjust viscosity with Sodium Chloride if necessary.

The resulting composition has a creamy, lubricating lather as it gently cleans and moisturizes the skin.

### Example 3

### Clear Mild Shampoo

| Phase | INCI (CTFA) Name/Chemical Name | Trade Name/Supplier Name | % | |
|---|---|---|---|---|
| 1 | Deionized Water | | Q.S. to 100.0 | |
| | Sodium Laureth Sulfate | STEOL ® CS-230 | 46.6 | (3-30% active) |
| | STEPAN ® SLL-FB (Sodium Lauroyl Lactylate) | | 3.0 | (0.2-30% active) |
| | STEPAN ® SSL-CG (Sodium Stearoyl | | 2.0 | (0.2-20% active) |
| | Lactylate) | | | |

| | |
|---|---|
| Hydrolyzed Wheat Protein (Croda) | 1.0 |
| DL-Panthenol (Roche) Panthenol | 0.5 |
| Preservative, Dye, Fragrance | q.s. |
| Citric Acid 50% solution | q.s. |
| Sodium Hydroxide 50% solution | q.s. |
| Sodium Chloride | q.s. |

### Procedure:

1. Charge vessel with Deionized Water. Start agitation.
2. Add STEOL® CS-230, STEPAN® SLL-FB and STEPAN® SSL-CG. Start heating to 160-165° F. Mix at 160-165° F. for 15-20 minutes.
3. Cool the batch to 80° F.
4. Add Hydrolyzed Whole Wheat Protein and DL-Panthenol. Mix well.
5. Add preservative, fragrance, and color, if desired.
6. Adjust pH to 5.5-6.5 with Citric acid or Sodium Hydroxide as necessary.
7. Adjust viscosity with Sodium Chloride.

The resulting composition provides rich, creamy foam and hair conditioning properties.

### Example 4

### Skin Lotion

| Phase | INCI (CTFA) Name/Chemical Name | Trade Name/Supplier Name | % | |
|---|---|---|---|---|
| 1 | Deionized Water | | Q.S. to 100.0 | |
| | (Sodium Stearoyl Lactylate) | Stepan ® SSL-CG | 1.0 | (0.1-10% active) |
| | (Isopropyl Palmitate) | Stepan IPP | 10.0 | (1-50% active) |
| | (Glycerol Monostearate) | Stepan GMS PURE | 3.0 | (0.1-10% active) |
| | (Cetyl Alcohol) | Stepan CETYL ALCOHOL, NF | 2.0 | (0.1-10% active) |

| | | |
|---|---|---|
| Borage Oil | Roche | 1.0 |
| Vitamin A Palmitate | Roche | 0.01 |
| dl-alpha-Tocopheryl Acetate | Roche | 0.01 |
| Preservative, Color, Dye | | q.s. |
| Citric Acid 50% | | q.s. |
| Sodium Hydroxide | | q.s. |

### Procedure:

1. Prepare water phase. Charge vessel with Deionized Water, add STEPAN® SSL-CG, start mixing and heating to 170-175° F.
2. Prepare oil phase. In a separate container, combine Stepan IPP, Stepan GMS pure and Stepan Cetyl Alcohol. Heat to 170-175° F.
3. Increase agitation of water phase and slowly add oil phase into the water phase. Emulsify for 20-25 minutes.
4. Start cooling to 80° F. Add Borage Oil, Vitamin A and Vitamin E. Mix.
5. Adjust pH with Citric Acid to 5.5-6.0 as necessary.
6. Add preservative, color and dye, if desired.

The resulting composition provides moisturization while leaving a velvety after-feel on skin.

### Example 5

### Syndet Soap Bar

Syndet Soap Bar Formulation

| | 3170-58C | 3170-58B |
|---|---|---|
| Sodium Cocoyl Isethionate | 45.00 | 45.00 |
| Stearic Acid | 25.00 | 25.00 |
| Sodium Tallow/Coco Soap | 10.00 | 10.00 |
| Sodium Isethionate | 5.00 | 5.00 |
| Stepan Mild SLL-FB | 10.00 | 0.00 |
| Stepan Mild SSL-CG | 0.00 | 10.00 |
| Water | 5.00 | 5.00 |
| | 100.00 | 100.00 |

### Procedure:

1. Weight out components.
2. Amalgamate in a one gallon pail (sodium stearoly lactylate (Stepan Mild SSL-CG) added in powdered form, sodium lauroly lactylate (Stepan Mild SSL-F8) melted at 80° C.)
3. Homogenize mixture in 3-roll mill.
4. Extrude using sigma extruder. Cold water was used to cool barrel of extruder to avoid making material too soft and liquid.
5. Heated extrusion cone (˜50° C.) used to facilitate extrusion of bars.

### Example 6

### Combo Soap Bar

Combo Soap Bar Formulation

| | 3170-81 |
|---|---|
| Sodium Tallow soap Base | 84.40 |
| Citric Acid | 0.05 |
| BHT | 0.05 |
| NaCl | 0.50 |
| Stepan Mild SLL-FB | 1.50 |
| Alpha Step BSS-45 | 1.50 |
| Water | 12.00 |
| | 100.00 |

### Procedure:

1. Heat neat soap up to ˜70° C.
2. Blend in Alpha Step BSS-45 and sodium lauroly lactylate until uniform using hydrofoil impeller at 300 ppm.
3. Spread neat soap mixture to a thin layer on a pyrex drying sheet.
4. Dry overnight in a 50° C. oven.
5. Roll-mill dried soap. Test moisture using Karl Fisher.
6. Adjust moisture to 12%. Roll-mill. Extrude and stamp into bars.

### Example 7

Example 7 is a formulation of a sodium-based surfactant system to which the sodium stearoyl lactylate of the present technology (Stepan® SSL-CG) has been added at a concentration of 2% by weight. The Stepan® SSL-CG sodium stearoly lactylate was determined by NMR analysis to have a fatty acid content of about 26% by weight, a stearoyl lactate (n=1) content of aout 60% by weight, a stearoyl lactylate (n>1) content of about 9% by weight, a lactic acid (n=0) content of about 4% by weight and a lactylic acid (n≧1) content of about 1.4% by weight. The formulation is set forth in Table A.

TABLE A
Example 7

| Ingredient | Wt. % Active |
|---|---|
| STEOL ® CS-230 | 12 |
| AMPHOSOL ® HCG | 3 |
| STEPAN ® SSL-CG | 2 |
| Water | 83 |

### Example 8

### Comparative

Example 8 is a formulation of the same sodium-based surfactant system used in Example 7, except that 2% by weight of a competitive sodium stearoyl lactylate was added to the surfactant system in place of the sodium stearoyl lactylate of the present technology. The competitive sodium stearoyl lactylate was determined by NMR analysis to have a fatty acid content of about 22% by weight, a stearoyl lactate (n=1) content of about 45% by weight, a stearoyl lactylate (n>1) content of about 22% by weight, a lactic acid (n=0) content of about 5% by weight, and a lactylic acid (n≧1) content of about 6% by weight.

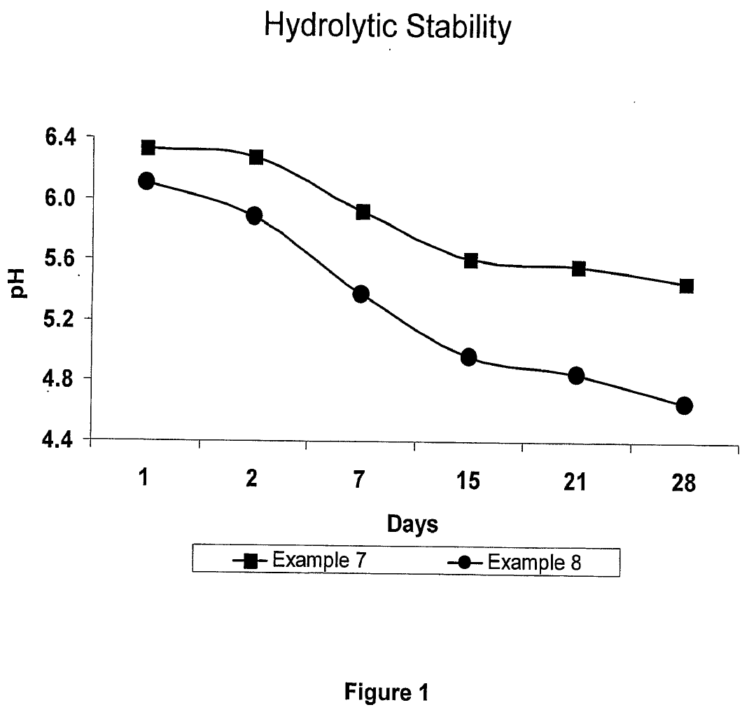

The Example 7 and Example 8 formulations were evaluated for hydrolytic stability by keeping the formulations at 50° C. over a four-week period. The hydrolytic stability of Example 7 and comparative Example 8 over the four-week period is graphically illustrated in FIG. 1.

As can be seen from FIG. 1, the Example 7 formulation containing the sodium stearoyl lactylate of the present technology maintained its pH within a range of about 5.5 to about 6.3 over the four-week period, whereas the Example 8 formulation containing the competitive sodium stearoyl lactylate had a steady drop in pH, down to about 4.6 by the end of the four-week period. The drop in pH for the Example 8 formulation demonstrates that the Example 8 formulation is not hydrolytically stable, whereas the Example 7 formulation, which maintained its pH within the range of about 6.3 to about 5.5, demonstrated enhanced hydrolytic stability. By “enhanced hydrolytic stability” is meant that the formulation exhibits a drop in pH of no more than about 1.0 pH unit at 50° C. over a four week period.

### Example 9

### Control Formulation

Example 9 is a control body wash formulation, for testing comparison, indicated in weight percent active.

### Example 9

| Ingredient | Wt. % Active |
|---|---|
| STEOL ® CS-230 | 12.0 |
| AMPHOSOL ® HCG | 3.0 |
| Water | 85.0 |

### Example 10

### Comparative

The control formulation from Example 9 was used as a base body wash formulation, and two different commercially available sodium stearoyl lactylates were each added in an amount of 2% by weight to the base body wash formulation to prepare competitive product 1 and competitive product 2 body wash formulations. Competitive product 1 comprised a sodium stearoyl lactylate comprising about 43% by weight stearoyl lactate (n=1), about 28% by weight stearoyl lactylate (n>1), about 20% by weight fatty acid, about 4% by weight lactic acid (n=0), and about 5% by weight lactylic acid (n≧1). Competitive product 2 comprised a sodium stearoyl lactylate comprising about 53% by weight stearoyl lactate (n=1), about 11% by weight stearoyl lactylate (n>1), about 21% by weight fatty acid, about 13% by weight lactic acid (n=0), and about 2.4% by weight lactylic acid (n≧1). The formulations are set forth below in Table B.

TABLE B
Example 10 (Comparative)

| | Example 10 | |
|---|---|---|
| | Wt. % Active | |
| Ingredient | Competitive Product 1 | Competitive Product 2 |
| STEOL ® CS-230 | 12 | 12 |
| AMPHOSOL ® HCG | 3 | 3 |
| SSL (competitor 1) | 2 | — |
| SSL (competitor 2) | — | 2 |
| Water | 83 | 83 |

The control body wash formulation of Example 9 and the Example 7 formulation, which comprises the same surfactant formulation as Example 9, with the addition of STEPAN® SSL-CG sodium stearoyl lactylate, were evaluated for foaming and skin-feel. In addition, the Example 10 (comparative) formulations, which are identical to the Example 7 formulation except that competitive sodium stearoyl lactylates were used in place of the STEPAN® SSL-CG product, were also evaluated for foaming and skin-feel. The foaming and skin-feel testing was performed using an in-vivo human expert panel test.

At least six panelists with different skin types (dry, normal, and moist) were chosen for each test. The skin type of the panelist was determined using a NOVA meter. A NOVA reading less than 100 represents dry skin, 110-130, normal skin and 130 or above, moist skin. The panelists were asked to assess the performance of the experimental product (Example 7) and the control (Example 9) or the commercial products (Example 10) in a blind test using a 1 to 5 rating scale, with 1 being the worst and 5 being the best. Panelists were not told which samples were the experimental formulation, and which samples were the control or commercial product.

Panelists were asked to assess the following characteristics during and after the washing procedure: foam volume, skin softness, skin dryness, and tackiness during drying. To identify tackiness during drying, the panelists were instructed that some products may impart a sticky/tacky feel on the skin during the transition from a wet to a dry stage. Tackiness can be assessed by touching the fingers of the same hand together or by force required to separate fingers. To identify skin tightness when dry, the panelists were instructed that some products may leave the skin feeling tight or stretched after the skin is completely dry. The panelists were instructed that this property should not be evaluated until the panelist is sure that the hands are completely dry. Similarly, skin dryness was evaluated once the hands were completely dry.

To identify skin softness, the panelists were instructed to characterize how soft and smooth the skin feels to the touch. A product can often leave the skin feeling dry, but smooth. The positive extreme would be a smooth velvety feel (ranking of 5 on a 1-5 scale), and the opposite would be a rough feeling skin with some grittiness (ranking of 1 on a 1-5 scale). All samples were coded in order to get a fair comparison between the experimental and control products.

### Human Panel Test Method

1. Panelists were asked to pre-wash their hands with 15% active sodium lauryl sulfate solution to remove residue from the skin and establish the baseline before evaluating of experimental body washes.
2. Hand washing tests were conducted using luke-warm (95° F. and 105° F.) running tap water.
3. Using a syringe, 1 ml of the test product was dispensed to the panelist's wet palm.
4. The panelists were asked to wash their hands by gently rubbing them together for 30 seconds followed by rinsing under running tap water for 15 seconds.
5. The washing procedure was repeated and the foam generated was collected and measured using a graduated beaker prior to rinsing.
6. The hands were rinsed for 15 seconds and dried using paper towel.
7. The panelists were asked to rank each product for ease of application, wet stage and dry stage (skin-feel) performance properties.
8. Skin feel evaluation was done at ambient temperature (˜25° C.).

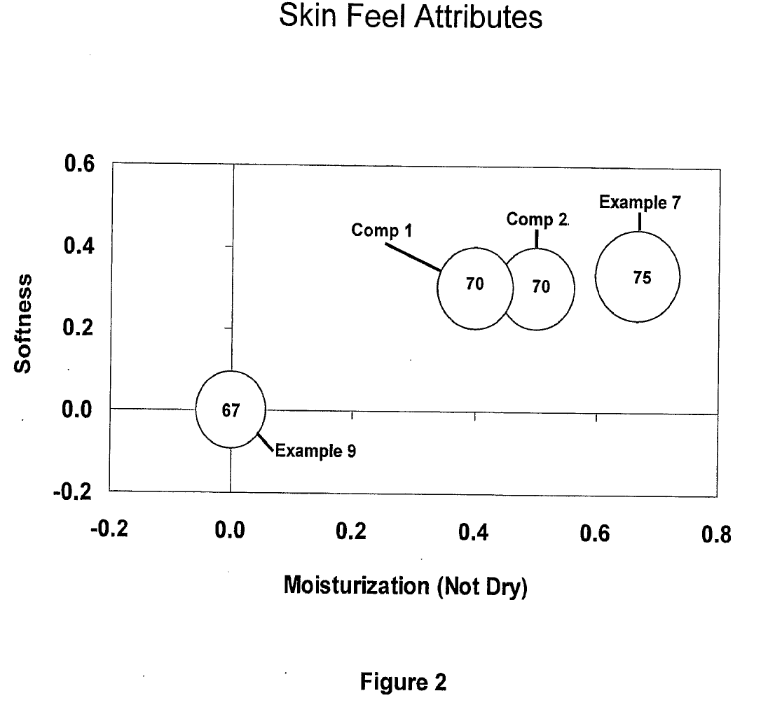

The average response for the panelists, for the experimental formulation, is subtracted from the average response for the control formulation. A positive score indicates that the experimental formulation outperformed the control. The results for the comparison of the experimental formulation of Example 7 with the control formulation of Example 9 and the comparative formulations of Example 10 are shown in the bubble chart of FIG. 2. As illustrated in FIG. 2, the formulation of Example 7, comprising the acyl lactylate in accordance with the present technology, gives improved skin-feel characteristics compared to the Example 9 control formulation and the Example 10 comparative formulations.

The volume of foam collected in the graduated beakers for each of the formulations was also measured and compared. The measured volume in ml is indicated in each of the bubbles shown in the FIG. 2 bubble chart. As illustrated in FIG. 2, the Example 7 formulation of the present technology had a foam volume of 75 ml, whereas the Example 10 comparative formulations and the Example 9 control formulation had foam volumes of only 70 ml and 67 ml, respectively.

The body wash formulation of Example 7 was also compared for performance properties with body wash formulations prepared with conventional polymeric additives on an equal cost contribution basis. Because conventional polymeric additives are typically more expensive than the presently described acy lactylates, lesser amounts of the polymeric additives were incorporated into the comparative body wash formulations in order to compare the performance properties on a more equal cost basis. The comparative body wash formulations are set forth below in Table C.

TABLE C
Polymeric Additive Body Wash Formulations

| | Comparative 1 | Comparative 2 | Comparative 3 |
|---|---|---|---|
| Ingredient | Wt. % Active | Wt. % Active | Wt. % Active |
| STEOL ® CS-230 | 12 | 12 | 12 |
| AMPHOSOL ® HCG | 3 | 3 | 3 |
| Polyquaternium-7 | 0.3 | — | — |
| (PQ-7) additive | | | |
| Polyquaternium 10 | — | 0.3 | — |
| (PQ-10) additive | | | |
| Guar Gum | — | — | 0.3 |
| Water | q.s. | q.s. | q.s. |

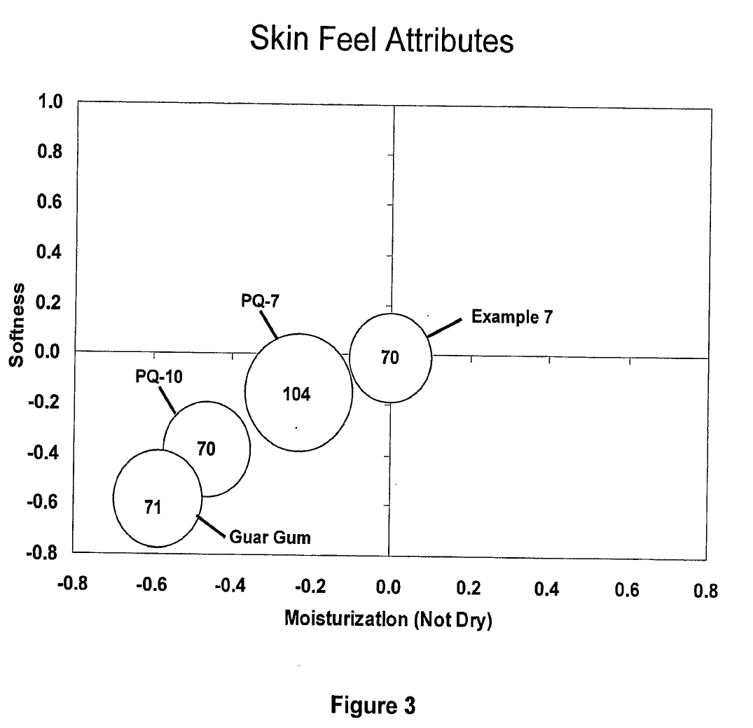

The Table C comparative body wash formulations and the Example 7 formulation were compared using the skin-feel test procedure described above. The results for the skin feel evaluation in terms of softness and moisturization are shown in the bubble chart of FIG. 3. As illustrated in FIG. 3, the formulation of Example 7 gives improved softness and moisturization compared to each of the Table C comparative formulations comprising a polymeric additive.

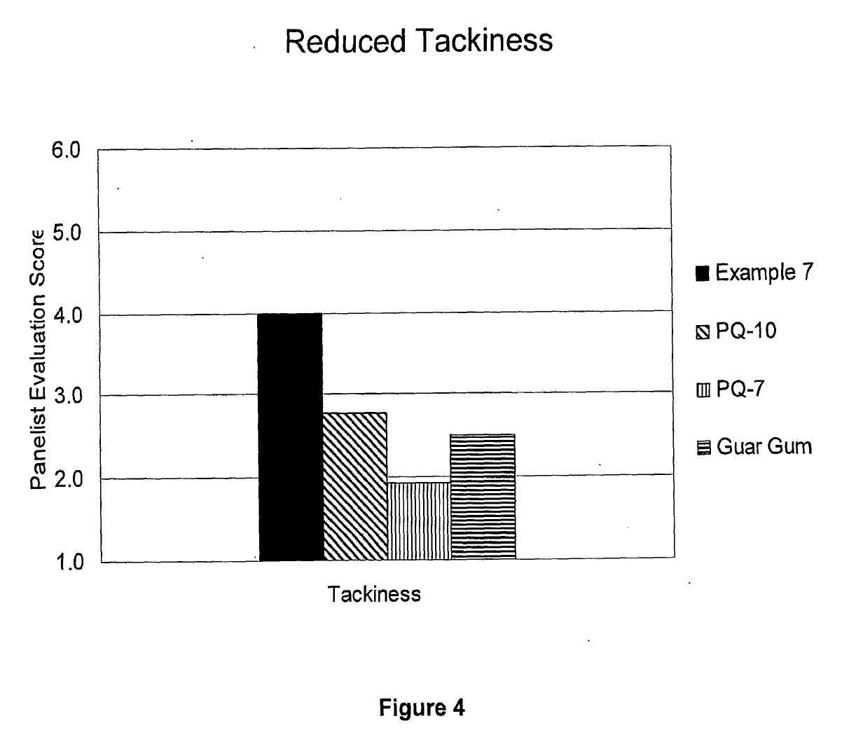

The results for the skin feel evaluation in terms of tackiness performance are shown in the bar graph of FIG. 4. Tackiness is indicated numerically with 1 being very tacky/sticky and 5 being not tacky. As illustrated in FIG. 4, the body wash formulation of Example 7 showed reduced tackiness compared to the Example 10 body wash formulations prepared with polymeric additives.

### Example 11

Example 11 is a model skin lotion formulation similar to the skin lotion formulation of Example 4, except that the borage oil and vitamins have been omitted. The Example 11 formulation is prepared following the procedure of Example 4. For comparison purposes, the Example 11 formulation was duplicated, except that a competitive sodium stearoyl lactylate was used in the comparative formulation rather than the Stepan® SSL-CG sodium stearoyl lactylate. The Example 11 and Example 11 (comparative) skin lotion formulations are set forth in Table D.

TABLE D
Example 11

| | Example 11 | Example 11 (comparative) |
|---|---|---|
| Ingredients | Wt % Active | Wt % Active |
| D.I. Water | Q.S. to 100.0 | Q.S. to 100.0 |
| STEPAN ® SSL-CG | 1.0 | — |
| (Sodium Stearoyl Lactylate) | | |
| Commercial SSL | — | 1.0 |
| STEPAN ® IPP | 10.0 | 10.0 |
| (Isopropyl Palmitate) | | |
| STEPAN ® GMS PURE | 3.0 | 3.0 |
| (Glycerol Stearate) | | |
| STEPAN ® Cetyl Alcohol | 2.0 | 2.0 |
| (Cetyl Alcohol) | | |
| Preservative | q.s. | q.s. |

Both formulations were adjusted with citric acid, as necessary, in order to have a final pH of 5.0. The viscosity of both formulations was measured. The Example 11 formulation, made with the Stepan® SSL-CF sodium stearoyl lactylate, had a viscosity of 10,000 cps, whereas the viscosity of the Example 11 (comparative) formulation was only 5,000 cps. Not only did the Example 11 lotion formulation have higher viscosity than the comparative formulation, photomicrographs of the formulations demonstrated that the Example 11 lotion had a better quality emulsion than the comparative formulation, with smaller and more uniform particle size.

### Example 12

A shampoo formulation was prepared by adding 2% by weight of sodium lauroyl lactylate (STEPAN® SLL-FB) to the control formulation of Example 9. The shampoo formulation is set forth in Table E.

TABLE E
Example 12 Shampoo Formulation

| Ingredient | Wt. % Active |
|---|---|
| STEOL ® CS-230 | 12 |
| AMPHOSOL HCG | 3 |
| STEPAN ® SLL-FB | 2 |
| Water | 83 |

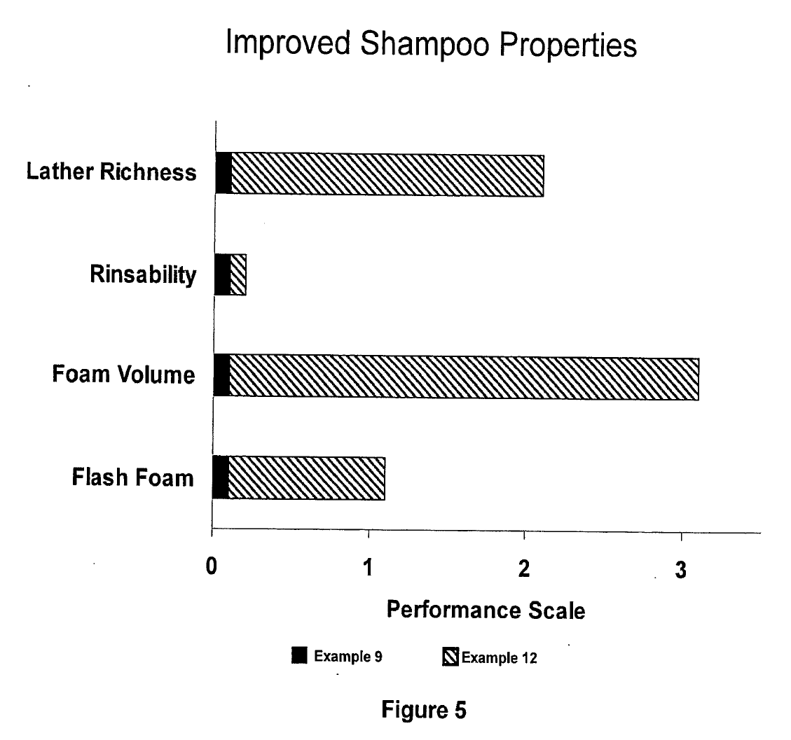

The performance properties of the Example 12 shampoo formulation were compared with the performance properties of the Example 9 control formulation in terms of lather richness, rinsability, foam volume, and flash foam. The results of the comparison are shown in the bar graph of FIG. 5. From FIG. 5, it can be seen that the addition of the sodium lauroyl lactylate composition of the present technology provides improved foaming, improved lather richness, and improved flash foam compared to the control formulation.

The presently described technology and the manner and process of making and using it, are now described in such full, clear, concise and exact terms as to enable one of ordinary skill in the art to which the present technology pertains, to make and use the same. It should be understood that the foregoing describes some embodiments and advantages of the invention and that modifications may be made therein without departing from the spirit and scope of the presently described technology as set forth in the claims. Moreover, the invention has been described with reference to preferred and alternate embodiments. Modifications and alterations will occur to others upon the reading and understanding of the specification. It is intended to include all such modifications and alterations insofar as they come within the scope of the appended claims or equivalents thereof. To particularly point out and distinctly claim the subject matter regarded as the invention, the following claims conclude this specification.

What is claimed is:

1. A rinse-out skin or hair composition comprising:
at least one acyl lactylate having an amount of lactylic acid of no more than about 2.0% by weight of the acyl lactylate;
at least one non-cationic surfactant; and
water.

2. The rinse-out skin or hair composition of claim 1, wherein the surfactant is selected from the group consisting of an anionic surfactant, an amphoteric surfactant, a non-ionic surfactant, and a zwitterionic surfactant.

3. The rinse-out skin or hair composition of claim 1, wherein the composition further comprises at least one fragrance, dye, protein, preservative, humectant, opacifying agent, thickener, pearlescent agent, derivatives thereof, or combinations thereof.

4. The rinse-out skin or hair composition of claim 1, wherein the composition is a shampoo, a body wash, a facial cleanser, a liquid hand soap, a bubble bath or a liquid body scrub.

5. The rinse-out skin or hair composition of claim 1, wherein the composition exhibits enhanced aqueous hydrolytic stability compared to acyl lactylates having an amount of lactylic acid of greater than about 2.0% by weight of the acyl lactylates.

6. The rinse-out skin or hair composition of claim 1, wherein the composition exhibits a pH stability in the range of from about 6.5 to about 5.5.

7. The rinse-out skin or hair composition of claim 1, wherein the composition exhibits an improved viscosity stability at 50° C. as a function of time compared to acyl lactylates having an amount of lactylic acid of greater than about 2.0% by weight of the acyl lactylates.

8. The rinse-out skin or hair composition of claim 1, wherein the composition exhibits improved foaming as compared to acyl lactylates having an amount of lactylic acid greater than about 2.0% by weight of the acyl lactylates.

9. The rinse-out skin or hair composition of claim 1, wherein the composition exhibits improved moisturization as compared to acyl lactylates having an amount of lactylic acid greater than about 2.0% by weight of the acyl lactylates.

10. A soap bar composition comprising:
at least one acyl lactylate having an amount of lactylic acid of no more than about 2.0% by weight of the acyl lactylate;
at least one non-cationic surfactant; and
water; and
wherein the soap bar composition has a moisture content of from about 5% to about 20%.

11. The soap bar composition of claim 10, wherein the moisture content is from about 8% to about 20%.

12. The soap bar of claim 10, wherein the surfactant is selected from the group consisting of an anionic surfactant, an amphoteric surfactant, a non-ionic surfactant, and a zwitterionic surfactant.

13. The soap bar of claim 10, wherein the composition further comprises at least one fragrance, dye, protein, preservative, humectant, opacifying agent, thickener, pearlescent agent, derivatives thereof, or combinations thereof.

14. The soap bar of claim 10, wherein the composition exhibits enhanced aqueous hydrolytic stability.

15. A leave-on skin or hair emulsion composition comprising:
at least one acyl lactylate having an amount of lactylic acid of no more than about 2.0% by weight of the acyl lactylate;
at least one non-cationic co-emulsifier and at least one emollient oil; and
water.

16. The leave-on skin or hair emulsion composition of claim 15, wherein the composition further comprises at least one fragrance, dye, protein, preservative, humectant, opacifying agent, thickener, pearlescent agent, derivatives thereof, or combinations thereof.

17. The leave-on skin or hair emulsion composition of claim 15, wherein the composition is a skin cream.

18. The leave-on skin or hair emulsion composition of claim 15, wherein the composition is a lotion.

19. The leave on skin or hair composition of claim 15, wherein the composition exhibits enhanced aqueous hydrolytic stability.

20. The leave on skin or hair composition of claim 15, wherein the composition exhibits improved foaming and moisturization as compared to acyl lactylates having an amount of lactylic acid of greater than about 2.0% by weight of the acyl lactylates.
